(19) United States
(12) Patent Application Publication
(10) Pub. No.: US 2022/0362222 A1
(43) Pub. Date: Nov. 17, 2022

(54) ONCE DAILY FORMULATIONS OF TACROLIMUS

(71) Applicant: TFF Pharmaceuticals, Inc., Fort Worth, TX (US)

(72) Inventors: Dale J. Christensen, Fort Worth, TX (US); Christopher S. Crean, Fort Worth, TX (US); John J. Koleng, Fort Worth, TX (US)

(21) Appl. No.: 17/742,945

(22) Filed: May 12, 2022

Related U.S. Application Data
(60) Provisional application No. 63/187,774, filed on May 12, 2021.

(51) Int. Cl. A61K 31/436; A61K 9/00; A61K 9/14; A61K 47/26

(52) U.S. Cl. CPC A61K 31/436; A61K 9/0075; A61K 9/14; A61K 47/26

(57) ABSTRACT

The present disclosure provides methods of using and pharmaceutical compositions of tacrolimus that may be administered by inhalation once a day. These methods and compositions may be used to generate a blood plasma concentration of tacrolimus in a therapeutically effective range using only a single dose daily. Administration once daily may result in few adverse events and reduced side effects while increasing patient compliance.

This application claims the benefit of priority to U.S. Provisional Application No. 63/187,774, filed on May 12, 2021, the entire contents of which are hereby incorporated by reference.

### BACKGROUND

### 1. Field

The present disclosure relates generally to the field of pharmaceuticals and pharmaceutical manufacture. More particularly, it concerns compositions and methods of administering tacrolimus in a once daily formulation.

### 2. Description of Related Art

Tacrolimus, an immunosuppressive drug, is used in transplant medicine. Tacrolimus is currently used in more than 80% of lung transplant patients based upon ISHLT data. Similarly high rates of tacrolimus use occur in heart, kidney and liver transplant patients. The high rate of usage of tacrolimus for immunosuppressive therapy occurs despite many challenges for patients and physicians when used for extended periods. Tacrolimus can cause toxicity in the kidneys, particularly when used in high doses.

Tacrolimus (Prograf®) is currently available as a solution injection, granule for suspension or gelatin coated capsule. None of the current formulations are suitable for inhalation. Prograf is indicated for the prophylaxis of organ rejection in adult and pediatric patients receiving allogeneic liver, kidney or heart transplants, in combination with other immunosuppressants. It is not indicated for prophylaxis in lung, heart and lung or double lung transplants. However, it is widely prescribed in lung transplant and recommended therapeutic drug monitoring concentrations, based in heart and lung transplantation, are between 15 to 20 ng/mL at C. (trough concentration) in the first weeks (Brunet et al., 2019) after transplant and then lower thereafter.

According to the Prograf® label, tacrolimus when administered orally, is administered twice a day in a dose range of 0.1-0.3 mg/kg/day. This is approximately 7-21 mg/day for a 70 kg human. Absorption of tacrolimus is incomplete (<25%) and variable (%CV range of 28-58%) depending on the study. Furthermore, absorption can be affected by food where the presence of food the decreased the rate and extent of absorption, which is most pronounced following a high-fat meal. Tacrolimus is also extensively metabolized by CYP3A4/5 in the liver and gut wall (Chen and Prasad, 2018) and thus drugs that are also metabolized by this CYP isozyme are known to interact with tacrolimus metabolism, such as ketoconazole, cyclosporine A, diltiazem, erythromycin and fluconazole (Iwasaki, 2007). Rifampicin can also decrease tacrolimus concentrations in kidney and liver transplant patient because it is an inducer of CYP3A4. Genotyping patients for CYP3A4 and CYP3A5 polymorphs helps improve targeting the initial dose and future dose adjustments but proper therapeutic drug monitoring is still a challenge.

One of the biggest challenges the medical community faces with tacrolimus is balancing patient compliance and monitoring for adverse events. There are a significant number of warnings and precautions with the use of Prograf® (oral tacrolimus), such as lymphoma, serious infections, new onset diabetes after transplant, nephrotoxicity, neurotoxicity, hyperkalemia, hypertension and QTc prolongation. However, adverse reactions associated with multiple daily oral administration may be a bigger concern. The observed adverse reaction rates can vary depending on the transplantation surgery and concomitant medication. Consistently, across studies, diarrhea is the primary GI adverse reaction with an incidence rate of 25% -72%, depending on the study; followed by nausea, vomiting and constipation (Prograf® label). In the case of lung transplant, to be able to bypass the GI and deliver tacrolimus directly to the target tissue may reduce this incidence and improve patient compliance. Such compositions that achieve the goal of reduce number of administrations while maintaining a therapeutically effective concentration of tacrolimus are highly sought after.

### SUMMARY

The present disclosure provides methods and compositions of tacrolimus that may be administered in a once daily formulation while achieving therapeutically effective concentrations throughout the 24 hour period. In some aspects, the present disclosure provides methods of modulating an immune response in a patient in need thereof, comprising administering by inhalation to the lungs of the patient an appropriate amount of a dry powder composition of drug particles comprising tacrolimus, the patient being administered a dose once during a 24 hour period sufficient to generate a blood concentration of tacrolimus in the patient of at least 3 ng/mL at a time point of 24 hours after the administration once the patient has been administered the dose for 3 consecutive days.

In some embodiments, the patient has been administered the dose for 7 consecutive days. In some embodiments, the patient has not been administered any additional tacrolimus other than the dose. In some embodiments, the dose is administered using a single inhaler capsule. In other embodiments, the dose is administered using multiple inhaler capsules of an appropriate amount on a single occasion.

In some embodiments, the blood concentration of tacrolimus is at least 5 ng/mL at a time point of 24 hours after the administration. In some embodiments, the blood concentration of tacrolimus is from about 3 ng/mL to about 15 ng/mL at a time point of 24 hours after the administration. In some embodiments, the blood concentration of tacrolimus is from about 3 ng/mL to about 12 ng/mL at a time point of 24 hours after the administration. In some embodiments, the blood concentration of tacrolimus is from about 3 ng/mL to about 7.5 ng/mL at a time point of 24 hours after the administration.

In some embodiments, the composition comprises a sugar such as lactose. In some embodiments, the drug particles comprise tacrolimus and the sugar in a weight ratio of 5:1 to about 1:20. In some embodiments, the weight ratio is from about 1:1 to about 1:10. In some embodiments, the weight ratio is from about 1:2.5 to about 1:10.

In some embodiments, the composition comprises a dose of tacrolimus from about 0.05 mg to about 3.5 mg. In some embodiments, the dose of tacrolimus is from about 0.1 mg to about 3.0 mg. In some embodiments, the dose of tacrolimus is from about 0.25 mg to about 2.5 mg. In some embodiments, the dose of tacrolimus is about 1.5 mg.

In some embodiments, the tacrolimus is in the amorphous form. In some embodiments, at least 90% of the tacrolimus is in the amorphous form. In some embodiments, at least 95% of the tacrolimus is in the amorphous form. In some embodiments, at least 98% of the tacrolimus is in the amorphous form. In some embodiments, at least 99% of the tacrolimus is in the amorphous form.

In some embodiments, the drug particles have a mass median aerodynamic diameter (MMAD) from about 0.5 μm to about 5.0 μm. In some embodiments, the MMAD is from about 1.0 μm to about 3.5 μm. In some embodiments, the MMAD is from about 1.5 μm to about 2.5 μm. In some embodiments, the drug particles have a geometric standard deviation (GSD) from about 0.5 to about 8. In some embodiments, the GSD is from about 1 to about 6. In some embodiments, the GSD is from about 2 to about 5.

In some embodiments, the composition of tacrolimus is loaded into a capsule. In some embodiments, the capsule is configured for use in an inhaler. In some embodiments, the composition is loaded into an inhaler. In some embodiments, the inhaler is a high resistance inhaler. In some embodiments, the inhaler is a dry powder inhaler.

In some embodiments, the drug particles when emitted from an inhaler has an emitted dose of at least 70%. In some embodiments, the emitted dose is at least 80%. In some embodiments, the emitted dose is at least 90%.

In some embodiments, the drug particles when emitted from an inhaler have a fine powder fraction as a percentage of the recovered dose of at least 40%. In some embodiments, the fine powder fraction as a percentage of the recovered dose is at least 45%. In some embodiments, the fine powder fraction as a percentage of the recovered dose is at least 50%. In some embodiments, the drug particles when emitted from an inhaler have a fine powder fraction as a percentage of the recovered dose is from about 40% to about 95%. In some embodiments, the fine powder fraction as a percentage of the recovered dose is from about 45% to about 90%. In some embodiments, the fine powder fraction as a percentage of the recovered dose is from about 50% to about 85%.

In some embodiments, the drug particles when emitted from an inhaler have a fine powder fraction as a percentage of the delivered dose of at least 50%. In some embodiments, the fine powder fraction as a percentage of the delivered dose is at least 55%. In some embodiments, the fine powder fraction as a percentage of the delivered dose is at least 60%. In some embodiments, the drug particles when emitted from an inhaler have a fine powder fraction as a percentage of the delivered dose is from about 50% to about 98%. In some embodiments, the fine powder fraction as a percentage of the delivered dose is from about 55% to about 95%. In some embodiments, the fine powder fraction as a percentage of the delivered dose is from about 60% to about 90%.

In some embodiments, the composition of tacrolimus is a tacrolimus and lactose composition comprising a dose of tacrolimus from about 0.1 mg to about 2.5 mg loaded into a capsule for use in an inhaler. In some embodiments, the modulation of the immune system is sufficient to prevent or slow rejection of a transplanted organ such as rejection of a transplanted kidney, heart, liver, or lung. In some embodiments, the modulation of the immune system is sufficient to suppress the patient's immune system such as by inhibiting of calcineurin.

In some embodiments, the patient is human. In some embodiments, the patient has been identified as a fast metabolizer of tacrolimus and the methods further comprise increasing the dose administered to the patient. In other embodiments, the patient has been identified as a slow metabolizer of tacrolimus and the methods further comprise decreasing the dose administered to the patient.

In another aspect, the present disclosure provides compositions for use in modulating an immune response in a patient, the composition having one or more drug particles comprising:
(A) a dose of tacrolimus; and
(B) a sugar;
   wherein the composition is formulated for administration to the lungs via inhalation, and the dose generates a blood concentration of tacrolimus in a patient of greater than 3 ng/mL at 24 hours after the dose has been administered once the patient has been administered the dose for 3 consecutive days.

In some embodiments, the patient has been administered the dose for 7 consecutive days. In some embodiments, the patient has not been administered tacrolimus since the administration. In some embodiments, the sugar is lactose.

In some embodiments, the blood concentration of tacrolimus is at least 5 ng/mL at a time point of 24 hours after the administration. In some embodiments, the blood concentration of tacrolimus is from about 3 ng/mL to about 15 ng/mL at a time point of 24 hours after the administration. In some embodiments, the blood concentration of tacrolimus is from about 3 ng/mL to about 12 ng/mL at a time point of 24 hours after the administration. In some embodiments, the blood concentration of tacrolimus is from about 3 ng/mL to about 7.5 ng/mL at a time point of 24 hours after the administration.

In some embodiments, the composition comprises a sugar such as lactose. In some embodiments, the drug particles comprise tacrolimus and the sugar in a weight ratio of 5:1 to about 1:20. In some embodiments, the weight ratio is from about 1:1 to about 1:10. In some embodiments, the weight ratio is from about 1:2.5 to about 1:10.

In some embodiments, the composition comprises a dose of tacrolimus from about 0.05 mg to about 3.5 mg. In some embodiments, the dose of tacrolimus is from about 0.1 mg to about 3.0 mg. In some embodiments, the dose of tacrolimus is from about 0.25 mg to about 2.5 mg. In some embodiments, the dose of tacrolimus is about 1.5 mg.

In some embodiments, the tacrolimus is in the amorphous form. In some embodiments, at least 90% of the tacrolimus is in the amorphous form. In some embodiments, at least 95% of the tacrolimus is in the amorphous form. In some embodiments, at least 98% of the tacrolimus is in the amorphous form. In some embodiments, at least 99% of the tacrolimus is in the amorphous form.

In some embodiments, the drug particles have a mass median aerodynamic diameter (MMAD) from about 0.5 μm to about 5.0 μm. In some embodiments, the MMAD is from about 1.0 μm to about 3.5 μm. In some embodiments, the MMAD is from about 1.5 μm to about 2.5 μm. In some embodiments, the drug particles have a geometric standard deviation (GSD) from about 0.5 to about 8. In some embodiments, the GSD is from about 1 to about 6. In some embodiments, the GSD is from about 2 to about 5.

In some embodiments, the composition of tacrolimus is loaded into a capsule. In some embodiments, the capsule is configured for use in an inhaler. In some embodiments, the composition is loaded into an inhaler. In some embodiments, the inhaler is a high resistance inhaler. In some embodiments, the inhaler is a dry powder inhaler.

In some embodiments, the drug particles when emitted from an inhaler has an emitted dose of at least 70%. In some embodiments, the emitted dose is at least 80%. In some embodiments, the emitted dose is at least 90%.

In some embodiments, the drug particles when emitted from an inhaler have a fine powder fraction as a percentage of the recovered dose of at least 40%. In some embodiments, the fine powder fraction as a percentage of the recovered dose is at least 45%. In some embodiments, the fine powder fraction as a percentage of the recovered dose is at least 50%. In some embodiments, the drug particles when emitted from an inhaler have a fine powder fraction as a percentage of the recovered dose is from about 40% to about 95%. In some embodiments, the fine powder fraction as a percentage of the recovered dose is from about 45% to about 90%. In some embodiments, the fine powder fraction as a percentage of the recovered dose is from about 50% to about 85%.

In some embodiments, the drug particles when emitted from an inhaler have a fine powder fraction as a percentage of the delivered dose of at least 50%. In some embodiments, the fine powder fraction as a percentage of the delivered dose is at least 55%. In some embodiments, the fine powder fraction as a percentage of the delivered dose is at least 60%. In some embodiments, the drug particles when emitted from an inhaler have a fine powder fraction as a percentage of the delivered dose is from about 50% to about 98%. In some embodiments, the fine powder fraction as a percentage of the delivered dose is from about 55% to about 95%. In some embodiments, the fine powder fraction as a percentage of the delivered dose is from about 60% to about 90%.

In some embodiments, the composition of tacrolimus is a tacrolimus and lactose composition comprising a dose of tacrolimus from about 0.1 mg to about 2.5 mg loaded into a capsule for use in an inhaler. In some embodiments, the modulation of the immune system is sufficient to prevent or slow rejection of a transplanted organ such as rejection of a transplanted kidney, heart, liver, or lung. In some embodiments, the modulation of the immune system is sufficient to suppress the patient's immune system such as by inhibiting of calcineurin.

In some embodiments, the patient is human. In some embodiments, the patient has been identified as a fast metabolizer of tacrolimus and the methods further comprise increasing the dose administered to the patient. In other embodiments, the patient has been identified as a slow metabolizer of tacrolimus and the methods further comprise decreasing the dose administered to the patient.

In still another aspect, the present disclosure provides methods of preventing organ rejection in a patient comprising administering to the patient in need thereof a therapeutically effective amount of a composition described herein.

In some embodiments, the organ rejection is rejection of a transplanted lung. UB other embodiments, the organ rejection is rejection of a transplanted heart. In other embodiments, the organ rejection is rejection of a transplanted kidney. In other embodiments, the organ rejection is rejection of a transplanted liver. In some embodiments, the methods comprise administering the composition once during a 24 hour time period.

In some embodiments, the methods comprise administering the composition as a single dose. In other embodiments, the methods comprise administering the composition as multiple doses at a single time.

In still another aspect, the present disclosure provides methods of modulating the immune system response in a patient in need thereof comprising administering to the patient a therapeutically effective amount of a composition described herein.

In some embodiments, the methods comprise administering the composition once during a 24 hour time period. In some embodiments, the methods comprise administering the composition as a single dose. In other embodiments, the methods comprise administering the composition as multiple doses at a single time.

In yet another aspect, the present disclosure provides compositions comprising:
(A) a dose of tacrolimus, wherein the dose is from about 0.1 mg to about 2.5 mg; and
(B) lactose;
   wherein the composition is formulated for administration via inhalation, the dose generates a blood concentration of tacrolimus in a patient of greater than 2 ng/mL at 24 hours after the dose has been administered, and the composition comprises a weight ratio of about 8:1 to about 12:1 lactose to the dose of tacrolimus. In some embodiments, the compositions have been formulated into a capsule for use in an inhaler or formulated into an inhaler.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows tacrolimus plasma exposure after inhalation of a 1 mg BID for 7 days.

FIGS. 2A & 2B show the plasma concentration of tacrolimus after the ^{1st} day and after the 7^{th} day plotted as a function of time for a 1.0 mg BID.

FIGS. 3A & 3B show the plasma concentration of tacrolimus after the 1^{st} day and after the 7^{th} day plotted as a function of time for a 1.0 mg BID along with simulated plasma concentration of tacrolimus for 1.0 mg and 1.5 mg SIM.

FIG. 4 show the simulated plasma concentration of tacrolimus for 1.0 mg and 1.5 mg SIM.

### DESCRIPTION OF ILLUSTRATIVE EMBODIMENTS

In some aspects of the present disclosure, the pharmaceutical compositions provided herein may comprise formulations of tacrolimus that may be administered once a daily while achieving a therapeutically effective blood concentration of the drug. These compositions may result in a sufficient blood concentration over a 24-hour period that a patient only needs to be administered a dose once. Reducing the number of doses may result in reduced side effects, reduced adverse events, and increased patient compliance. For example, the compositions may result in decreased renal toxicity, creatinine levels in the blood, or blood urea nitrogen levels or increased glomerular filtration rate.

Also provided herein are methods of preparing and using these compositions. Details of these compositions are provided in more detail below.

### I. PHARMACEUTICAL COMPOSITIONS

In some aspects, the present disclosure provides pharmaceutical compositions containing an active agent, such as tacrolimus and may contain an excipient. Theses composition may be formulated for administration via inhalation such inhalation may be to the lungs. The administration of tacrolimus may be as a single dose once day as a single capsule, or, alternatively, administered as multiple capsules administered at a single time. Furthermore, these pharmaceutical compositions may contain one or more properties that allow them to be delivered to the lungs through an inhaler. These particles show enhanced ability to break into smaller components. The particles may show a high surface area, a low tapped density, or a low bulk density. The surface area of the particles may be greater than 10 m²/g, greater than 25 m²/g, or greater than 50 m²/g. The bulk density of the particles may be less than 1 g/mL, less than 0.5 g/mL, or less than 0.25 g/mL. Finally, the tapped density of the particles may be less than 0.1 g/cm³, 0.05 g/cm³, or 0.025 g/cm³. Furthermore, these compositions may show improved flowability or compressibility such as a low Carr's Index such as less than 20, less than 15, or less than 10.

These compositions may be used to achieve a therapeutically effective plasma blood concentration, or simply blood concentration, for a time period of at least 18 hours, 20 hours, 22 hours, 24 hours, or 26 hours. In some embodiments, the composition administered herein may achieve a blood concentration of at least 2 ng/mL in a human for the time period. The blood concentration may be at least 2 ng/mL, at least 3 ng/mL, at least 4 ng/mL, or at least 5 ng/mL. The composition may achieve a blood concentration from about 2 ng/mL to about 15 ng/mL, from about 3 ng/mL to about 12 ng/mL, or from about 3 ng/mL to about 7.5 ng/mL. The blood concentration of tacrolimus may be from about 2 ng/mL, 3 ng/mL, 4 ng/mL, 5 ng/mL, 6 ng/mL, 7.5 ng/mL, 8 ng/mL, 10 ng/mL, 12 ng/mL, 12.5 ng/mL, 14 ng/mL, 16 ng/mL, 18 ng/mL, 20 ng/mL, 22 ng/mL, 24 ng/mL, to about 25 ng/mL, or any range derivable therein. The blood plasma concentration is determined by either LCMS or by ELISA. This blood concentration is obtained after administering the composition to the patient for 2, 3, 4, 5, 6, 7, or 14 days.

A. Tacrolimus

The pharmaceutical compositions described herein comprise tacrolimus as an active agent. The pharmaceutical compositions described herein contain tacrolimus in an amount between about 1% to about 25% w/w, between about 2% to about 20% w/w, between about 5% to about 15% w/w, or between about 7.5% to about 12.5% w/w of the total composition. In some embodiments, the amount of the tacrolimus is from about 1%, 2%, 3%, 4%, 5%, 6%, 7.5%, 8%, 10%, 12%, 12.5%, 14%, 15%, 20%, to about 25% w/w or any range derivable therein. In some embodiments, the pharmaceutical composition has a dose of tacrolimus from about 0.05 mg to about 3.5 mg, from about 0.1 mg to about 3.0 mg, or from about 0.25 mg to about 2.5 mg. The dose of tacrolimus may be from about 0.05 mg, 0.1 mg, 0.25 mg, 0.5 mg, 0.75 mg, 1.0 mg, 1.25 mg, 1.5 mg, 1.75 mg, 2.0 mg, 2.25 mg, 2.5 mg, 2.75 mg, 3.0 mg, 3.25 mg, to about 3.5 mg, or any range thereof.

In some aspects, a wide variety of different forms of tacrolimus may be used. Tacrolimus is an active agent with a chemical name of (1R,9S,12S,13R,14S,17R,18E,21S,23S,24R,25S,27R)-1,14-dihydroxy-12-[(E)-1-[(1R,3R,4R)-4-hydroxy-3 -methoxycyclohexyl]prop-1-en-2-yl]-23 ,25-dimethoxy-13,19,21,27-tetramethyl-17-prop-2-enyl-11,28 -dioxa-4-azatricyclo [22.3.1.0^{4.8}]octacos-18-ene-2,3,10,16-tetrone. Tacrolimus is sold as brand names Prograf®, Protopic®, Advagraf®, Envarsus XR®, and Chiesi®, but is also known as fujimycin with a CAS No. of 104987-11-3. Tacrolimus is an immunosuppressive drug used to reduce the risk of organ rejection. Furthermore, tacrolimus may also be used in other indications where immunomodulation may be needed. Acting through inhibition of calcineurin, the compound, a macrolide lactone, modulates the production of interleukin-2. Tacrolimus is metabolized by Cytochrome P450 and thus modifications to this enzyme or other compounds which modulate the enzyme's activity are known to lead changes in metabolism of tacrolimus. Therefore, patients with mutations in the CYP3A4/5 enzyme should be genotyped to determine the effect on tacrolimus dosing. If a patient is a fast metabolizer, then the dose may need to be increased. Conversely, if the patient is a slow metabolizer, then the dose may need to be decreased. Similarly, if the patient is taking another API that modulates CYP3A4/5 or eating a diet rich in foods that modulate this enzyme may need to have their dose of tacrolimus modulated.

In some embodiments, the particles comprise at least 80% of tacrolimus in the amorphous phase. In some embodiments, the amount of tacrolimus in the amorphous phase is from about 80%, 85%, 90%, 95%, 96%, 97%, 98%, 99%, 99.1%, 99.2%, 99.3%, 99.4%, 99.5%, 99.6%, 99.7%, 99.8%, to about 99.9%, or any range derivable therein.

In some embodiments, the drug particles have a mass median aerodynamic diameter from about 0.5 μm to about 5.0 μm, from about 1.0 μm to about 3.5 μm, or from about 1.5 μm to about 2.5 μm. In some embodiments, the drug particles have a mass median aerodynamic diameter from about 0.5 μm, 0.6 μm, 0.8 μm, 1.0 μm, 1.2 μm, 1.4 μm, 1.5 μm, 1.6 μm, 1.8 μm, 2.0 μm, 2.2 μm, 2.4 μm, 2.5 μm, 2.6 μm, 2.8 μm, 3.0 μm, 3.2 μm, 3.4 μm, 3.5 μm, 4.0 μm, 4.5 μm, to about 5.0 μm, or any range derivable therein. Copley Inhaler Testing Data Analysis Software (CITDAS) version 3.10 (Copley Scientific, Nottingham, UK) was used to calculate the aerodynamic particle size distribution including mass median aerodynamic diameter (MMAD), fine particle fraction (FPF), geometric standard deviation (GSD) and emitted fraction (EF). Mass median aerodynamic (MMAD) and geometric standard deviation (GSD) were evaluated by the cumulative percentage of mass and the aerodynamic diameter.

In some embodiments, the drug particles have a geometric standard deviation (GSD) from about 0.5 to about 8, from about 1 to about 6, or form about 2 to about 5. In some embodiments, the particles containing tacrolimus have a geometric standard deviation (GSD) from about 0.5, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8 to about 8.0, or any range derivable therein.

In some embodiments, the drug particles when loaded into an inhaler has a fine particle fraction of recovered dose is greater than 40%, greater than 45%, or greater than 50%. In some embodiments, the drug particles a fine particle fraction of recovered dose is greater than 40%, 41%, 42%, 43%, 44%, 45%, 46%, 47%, 48%, 49%, 50%, 51%, 52%, 53%, 54%, 55%, 56%, 57%, 58%, 59%, or 60%. In some embodiments, the drug particles have a fine powder fraction of recovered dose from about 40% to about 95%, from about 45% to about 90%, or from about 50% to about 85%. In some embodiments, the fine powder fraction of delivered dose is from about 40%, 45%, 50%, 55%, 60%, 65%, 70%, 75%, 80%, 85%, 90%, 95%, 96%, 97%, 98%, 99%, to about 99.5%, or any range derivable therein. The fine particle fraction of recovered dose was calculated from a fine-particle dose divided by a total mass (recovered dose) while a fine particle fraction of delivered dose was calculated from a fine-particle dose divided by a delivered dose. The fine particle dose and fraction was calculated at a 5 μm cutoff. Moreover, the percentage recovery was calculated by a percentage of a total mass (recovered dose) that was collected through NGI divided by a loading dose.

In some embodiments, the drug particles when loaded into an inhaler have a fine particle fraction of delivered dose is greater than 50%, greater than 55%, or greater than 60%. In some embodiments, the drug particles have a fine particle fraction of delivered dose is greater than 40%, 41%, 42%, 43%, 44%, 45%, 46%, 47%, 48%, 49%, 50%, 51%, 52%, 53%, 54%, 55%, 56%, 57%, 58%, 59%, or 60%. In some embodiments, the drug particles have a fine powder fraction of delivered dose from about 50% to about 98%, from about 55% to about 95%, or from about 60% to about 90%. In some embodiments, the fine powder fraction of delivered dose is from about 40%, 45%, 50%, 55%, 60%, 65%, 70%, 75%, 80%, 85%, 90%, 95%, 96%, 97%, 98%, 99%, to about 99.5%, or any range derivable therein.

In some embodiments, the drug particles when loaded into an inhaler have an emitted fraction as measured by an NGI greater than 70%, greater than 80%, or greater than 90%. In some embodiments, the pharmaceutical compositions have an emitted fraction of the particles containing tacrolimus as measured by an NGI greater than 70%, 75%, 80%, 81%, 82%, 83%, 84%, 85%, 86%, 87%, 88%, 89%, 90%, 91%, 92%, 93%, 94%, or 95%. The emitted fraction (EF) was calculated as the total amount of the emitted dose from the device as a percentage of the total amount that was collected through NGI.

1. Inhalation

In some embodiments, the present disclosure relates to respirable particles must be in the aerodynamic size range of around 0.5 to 5 microns or 0.5 to 3 microns in aerodynamic diameter. Typical approaches to obtain particles of this size range is by air jet milling, spray drying, thin-film freezing, and other methods known in the art. Drug particles are admixed by blending with appropriate carrier(s) particles using conventional mixing, or the drug and carrier can be prepared simultaneously from a solution or suspension formulation such as by spray-drying or thin film freezing processes.

In some embodiments, the present disclosure provides methods for the administration of the inhalable tacrolimus composition provided herein using a device. Administration may be, but is not limited, to inhalation of tacrolimus using an inhaler. In some embodiments, an inhaler is a simple passive dry powder inhaler (DPI), such as a Plastiape RSO1 monodose DPI. In a simple dry powder inhaler, dry powder is stored in a capsule or reservoir and is delivered to the lungs by inhalation without the use of propellants.

In some embodiments, an inhaler is a single-dose DPI, such as a DoseOne™ Spinhaler, Rotahaler®, Aerolizer®, or Handihaler. In some embodiments, an inhaler is a multidose DPI, such as a Plastiape RS02, Turbuhaler®, Twisthaler™, Diskhaler®, Diskus®, or Ellipta™. In some embodiments, the inhaler is Twincer®, Orbital®, TwinCaps®, Powdair, Cipla Rotahaler, DP Haler, Revolizer, Multi-haler, Twister, Starhaler, or Flexhaler®. In some embodiments, an inhaler is a plurimonodose DPI for the concurrent delivery of single doses of multiple medications, such as a Plastiape RS04 plurimonodose DPI. Dry powder inhalers have medication stored in an internal reservoir, and medication is delivered by inhalation with or without the use of propellants. Dry powder inhalers may require an inspiratory flow rate greater than 30 L/min for effective delivery, such as between about 30-120 L/min.

In some embodiments, the inhalable tacrolimus is delivered as a propellant formulation, such as HFA propellants.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator. The medication formulation, including propellants and any required excipients, are stored in the canister. The metering valve allows a defined quantity of the medication formulation to be dispensed. The actuator of the metered dose inhaler, or mouthpiece, contains the mating discharge nozzle and typically includes a dust cap to prevent contamination.

In some embodiments, the composition may be administered on a routine schedule. As used herein, a routine schedule refers to a predetermined designated period of time. The routine schedule may encompass periods of time which are identical, or which differ in length, as long as the schedule is predetermined. For instance, the routine schedule may involve administration every day, every two days, every three days, every four days, every five days, every six days, a weekly basis, a monthly basis or any set number of days or weeks therebetween. Alternatively, the predetermined routine schedule may involve administration on a twice daily basis for the first week, followed by a daily basis for several months, etc. In some embodiments, tacrolimus is administered once per day. In some embodiments, a complete dose of tacrolimus is between 0.05-5 mg, such as 0.1-2.5, 0.25-2, 0.5-1.5, or 1-1.5 mg.

In some embodiments, tacrolimus may be provided in a unit dosage form, such as in a capsule, blister or a cartridge, wherein the unit dose comprises at least 0.1 mg of tacrolimus, such as at least 0.1 mg, 1.5 mg or 2.5 mg of tacrolimus per dose. In particular aspects, the unit dosage form does not comprise the administration or addition of any excipient and is merely used to hold the powder for inhalation (i.e., the capsule, blister, or cartridge is not administered). In some embodiments, tacrolimus may be administered in a high emitted dose, such as at least 0.1 mg, preferably at least 1.5 mg, more preferably 2.5 mg. In some embodiments, administration of tacrolimus results in a high fine particle dose into the deep lung such as greater than 0.25 mg. Preferably, the fine particle dose into the deep lung is at least 0.5 mg, even more preferably at least 1.5 mg.

In some embodiments, changes in pressure drop across the device result in a change in emitted dose. In some embodiments, changes in pressure drop across the device of 3 kPa, such as from 4 kPa to 1 kPa, result in a reduction of emitted dose of less than 35%, such as 34%, 33%, 32%, 31%, 30%, 29%, 28%, 27%, 26%, 25%, 24%, 23%, 22%, 21%, 20%, 19%, 18%, 17%, 16%, 15% or less. In some embodiments, changes in inhalation pressure drop across the device result in a change in fine particle dose. In some embodiments, changes in inhalation pressure drop across the device of 3 kPa, such as from 4kPa to 1 kPa result in a reduction of fine particle dose of less than 35%, such as 34%, 33%, 32%, 31%, 30%, 29%, 28%, 27%, 26%, 25%, 24%, 23%, 22%, 21%, 20%, 19%, 18%, 17%, 16%, 15% or less.

2. Uses of Compositions

Tacrolimus (TAC) is a widely used immunosuppressive agent isolated from Streptomyces tsukubaensis. It has proven to be a potent immunosuppressant in transplantation medicine for treatment of organ rejection and different immunological diseases such as pulmonary fibrosis and bronchiolar asthma. TAC was first introduced as rescue therapy when cyclosporin A (CsA) therapy failed to prevent graft rejection. It has a mechanism of action similar to that of CsA, but its immunosuppressive activity is 10- to 100-times more potent than CsA. TAC is currently available in both an intravenous and oral dosage form (commercially known as Prograf®). However, these current available dosage forms of the drug are poorly tolerated and provide a variable and/or low bioavailability. The oral formulations of TAC present a considerable challenge as the drugs are practically insoluble in water and extensively metabolized from both CYP3A4 metabolism and p-glycoprotein efflux transport within the intestinal epithelium. The oral bioavailability of TAC varies from 4% to 93%. Inefficient or erratic drug absorption is primarily the result of incomplete absorption from the gastrointestinal tract and first-pass metabolism, which is subject to considerable inter-individual variation.

A. Lung Transplants

Pulmonary diseases continue to increase and are currently among the leading causes of death. Lung transplantation, introduced in the 1980s, has matured into successful therapy for select patients with end-stage lung disease. Immunosuppression has been a key factor for the success of organ transplants; the advent of cyclosporine brought about significant improvements in patient survival (Calne et al., 1978). The first successful lung transplants used an en bloc technique with a tracheal anastomosis which evolved to a more natural transplantation method that avoids cardiopulmonary bypass (if needed) (Patterson et al., 1988). This technique is the standard practice today for double-lung transplants.

The indications for lung transplantation can be broadly separated into the following main categories of end-stage lung diseases: obstructive lung disease, septic lung disease, fibrotic lung disease, and vascular lung disease. Of these categories, chronic obstructive pulmonary disease (COPD), cystic fibrosis (CF), interstitial pulmonary fibrosis (IPF), and primary pulmonary arterial hypertension make up the most common indication in each category, respectively (Christie et al., 2012). Lung transplantation for pulmonary malignancy has also been shown to be effective in highly selected patients (Machuca et al., 2012).

The current International Society for Heart and Lung Transplantation (ISHLT) and also the American Thoracic Society (ATS) selection criteria include appropriate age, clinically and physiologically severe disease, ineffective or unavailable medical therapy, substantial limitations in activities of daily living, limited life expectancy, adequate cardiac function without significant coronary disease, ambulatory with rehabilitation potential, acceptable nutritional status, and satisfactory psychosocial profile and emotional support system (Table 1). Patients who undergo lung transplantation subject themselves to lifelong immunosuppression and surveillance. Thus, candidates for lung transplantation who have smoking or drug dependency, psychiatric issues affecting compliance with postoperative care, or absence of a reliable social support network will generally not be listed for lung transplantation. Previous malignancy, particularly in the 2 years leading up to potential transplantation, is also a contraindication because of the need for lifelong immunosuppression. Indeed, although immunosuppression will potentiate infection, chronic infection is an intrinsic part of septic lung diseases and creates a challenge in these patients. In cystic fibrosis patients, nontuberculous mycobacterial, multi-drug-resistant bacteria, and Aspergillus species are commonly isolated (Helmi et al., 2003; Gilljam et al., 2010). An effort is made to eradicate infection or at least minimize colonization with these organisms before and following transplantation; the use of immunosuppressants will only exacerbate existing infections.

Patients requiring a lung transplant are placed upon a transplant waitlist. Because demand exceeds supply for lung transplants, a strategy for selecting patients based upon the need of a patient for a lung transplant and the probability of post-transplant survival was developed (Egan et al., 2006). The Lung Allocation Score (LAS) is calculated using statistical models based upon the patient's clinical and physiological characteristics, along with a measure of urgency (expected number of days lived without a transplant during an additional waitlist year) and a measure of survival following transplantation (expected number of days lived in the first year posttransplant). The urgency measure is subtracted from the benefit measure and then normalized to give an LAS. Patients with higher scores are allocated lungs sooner. Since the introduction of the LAS system, waitlist times have decreased, and the number of transplants has increased. Moreover, LAS scores have gradually increased, representing the increasing urgency of patients getting listed.

Although lung transplantation has been shown to confer increased survival to patients with end-stage lung disease, survival following lung transplantation is approximately 80% at 1 year, but still only 50% at 5 years (Christie et al., 2010). The major causes of death following lung transplantation vary with the time following transplantation. Thirty-day mortality is generally related primarily to surgical issues, donor lung preservation issues, and primary graft dysfunction (PGD) (Studer et al., 2004). Infectious causes, malignancy, and chronic lung allograft dysfunction (CLAD) predominate in the subsequent post-transplant period. Despite significant improvements in short-term outcomes owing to improved donor organ preservation and surgical technique, the long-term survival after lung transplantation remains at around a 50% 5-yr survival mostly because of the development of CLAD (Christie et al., 2010).

Lung transplant immunosuppression generally consists of a three-drug regimen with the exact composition being center dependent. A calcineurin inhibitor (cyclosporine or tacrolimus), a nucleotide blocking agent (azathioprine or mycophenolate mofetil), and corticosteroids make up the three drugs. Induction therapy, the use of a potent immunosuppression agent to deplete T cells such as anti-IL2R antibodies, anti-CD52 antibodies, or antithymocyte globulin, is controversial in lung transplantation. Immunosuppression therapy is required for life.

According to the 2012 ISHLT Registry report, tacrolimus is the most frequently used calcineurin inhibitor, with 83% of patients receiving tacrolimus at 1-year post-transplant and 77% receiving tacrolimus at 5 years post-transplant (Christie et al., 2012).

B. Excipients

In some aspects, the present disclosure comprises one or more excipients formulated into pharmaceutical compositions. An “excipient” refers to pharmaceutically acceptable carriers that are relatively inert substances used to facilitate administration or delivery of an active pharmaceutical ingredient (API) into a subject or used to facilitate processing of an API into drug formulations that can be used pharmaceutically for delivery to the site of action in a subject. Non-limiting examples of excipients include stabilizing agents, surfactants, surface modifiers, solubility enhancers, buffers, encapsulating agents, antioxidants, preservatives, nonionic wetting or clarifying agents, viscosity increasing agents, and absorption-enhancing agents.

In some aspects, the amount of the excipient in the pharmaceutical composition is from about 40% to about 99% w/w, from about 50% to about 98% w/w, from about 60% to about 96% w/w, or from about 75% to about 95% w/w. The amount of the excipient in the pharmaceutical composition comprises from about 40%, 45%, 50%, 55%, 60%, 65%, 70%, 75%, 80%, 85%, 90%, 92.5%, 95%, 96%, 98%, to about 99% w/w, or any range derivable therein, of the total pharmaceutical composition. In one embodiment, the amount of the excipient in the pharmaceutical composition is at 75% to 95% w/w of the total weight of the pharmaceutical composition. The weight ratio of the tacrolimus and the sugar in the composition is from 5:1 to about 1:20, from about 1:1 to about 1:10, or from about 1:2.5 to about 1:10.

In some aspects, the present disclosure may further comprise one or more excipient such as a saccharide or amino acid. Some composition may further comprise a mixture of two or more excipients including two or more saccharides or amino acids.

1. Saccharides and Amino Acids

In some aspects, the present disclosure comprises one or more excipients formulated into pharmaceutical compositions. In some embodiments, the excipients used herein are water soluble excipients. These water-soluble excipients include carbohydrates or saccharides such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In other aspects, larger molecules like amino acids, peptides and proteins are incorporated to facilitate inhalation delivery, including leucin, trileucine, histidine and others.

### II. manufacturing methods

A. Thin Film Freezing

Thus, in one aspect, the present disclosure provides pharmaceutical compositions which may be prepared using a thin-film freezing process. Methods of preparing pharmaceutical compositions using thin film freezing are described in U.S. Patent Application No. 2010/0221343, Watts, et al., 2013, Engstrom et al. 2008, Wang et al. 2014, Thakkar at el. 2017, O'Donnell et al. 2013, Lang et al. 2014a, Lang et al. 2014b, Carvalho et al. 2014, Beinborn et al. 2012a, Beinborn et al. 2012b, Zhang et al. 2012, Overhoff et al .2009, Overhoff et al.2008, Overhoff et al. 2007a, Overhoff et al. 2007b, Watts et al. 2010, Yang et al. 2010, DiNunzio et al. 2008, Purvis et al. 2007, Liu et al. 2015, Sinswat et al. 2008, and U.S. Pat. No. 8,968,786, all of which are incorporated herein by reference. In some embodiments, these methods involve dissolving the components of the pharmaceutical composition into a solvent to form a pharmaceutical mixture. The solvents may be either water or an organic solvent. Some non-limiting examples of organic solvents which may be used include volatile organic solvent such as 1,4-dioxane, acetonitrile, acetone, methanol, ethanol, isopropanol, dichloromethane, chloroform, tetrahydrofuran, tert-butyl alcohol, dimethyl sulfoxide, N,N-dimethyl formamide, diethyl ether, ethyl acetate, isopropyl acetate, butyl acetate, propyl acetate, toluene, hexanes, heptane, pentane, or combinations thereof. In some embodiments, the pharmaceutical mixture may contain less than 100 mg/mL of the therapeutic agent and excipient. The pharmaceutical mixture may contain less than 100, 90, 80, 70, 60, 50, 40, 30, 20, 17.5, 15, 12.5, 10, 7.5, 5, 2.5, or 1 mg/mL, or any range derivable therein.

This pharmaceutical mixture may be deposited on a surface which is at a temperature that causes the pharmaceutical mixture to freeze. In some embodiments, this temperature may be below the freezing point of the solution at ambient pressure. In other embodiments, a reduced pressure may be applied to the surface causing the solution to freeze at a temperature below the ambient pressure's freezing point. The surface may also be rotating or moving on a moving conveyer-type system thus allowing the pharmaceutical mixture to distribute evenly on the surface. Alternatively, the pharmaceutical mixture may be applied to surface in such a manner to generate an even surface.

After the pharmaceutical mixture has been applied to the surface, the solvent may be removed to obtain a pharmaceutical composition. Any appropriate method of removing the solvent may be applied including evaporation under reduced pressure or elevated temperature or lyophilization. In some embodiments, the lyophilization may comprise a reduced pressure and/or a reduced temperature. Such a reduced temperature may be from 25° C. to about −200° C., from 20° C. to about −175° C., from about 20° C. to about −150° C., from 0° C. to about −125° C., from −20° C. to about −100° C., from −75° C. to about −175° C., or from −100° C. to about −160° C. The temperature is from about −20° C., −30° C., −35° C., −40° C., −45° C., −50° C., −55° C., −60° C., −70° C., −80° C., −90° C., −100° C., −110° C., −120° C., −130° C., −140° C., −150° C., −160° C., −170° C., −180° C., −190° C., to about −200° C., or any range derivable therein. Additionally, the solvent may be removed at a reduced pressure of less than 500 mTorr, 450 mTorr, 400 mTorr, 375 mTorr, 350 mTorr, 325 mTorr, 300 mTorr, 275 mTorr, 250 mTorr, 225 mTorr, 200 mTorr, 175 mTorr, 150 mTorr, 125 mTorr, 100 mTorr, 75 mTorr, 50 mTorr, or 25 mTorr, or removed at a reduced pressure at any range of pressures derivable therein.

Such as composition prepared using these methods may exhibit a brittle nature such that the composition is easily sheared into smaller particles when processed through a device. These compositions have a high skeletal density and have high surface areas as well as exhibit improved flowability of the composition. Such flowability may be measured, for example, by the Carr index or other similar measurements. In particular, the Carr's index may be measured by comparing the bulk density of the powder with the tapped density of the powder. Such compounds may exhibit a favorable Can index and may result in the particles being better sheared to give smaller particles when the composition is processed through a secondary device to deliver the drug.

### III. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified polysaccharide, wherein at least one of the monomeric saccharide units is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “dissolution” as used herein refers to a process by which a solid substance, here the active ingredients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pp. 2).

As used herein, “inhalation” or “pulmonary inhalation” is used to refer to administration of pharmaceutical preparations by inhalation so that they reach the lungs and in particular embodiments the alveolar regions of the lung. Typically, inhalation is through the mouth, but in alternative embodiments in can entail inhalation through the nose.

As used herein, “dry powder” refers to a fine particulate composition that is not suspended or dissolved in an aqueous liquid.

A “simple dry powder inhaler” refers a device for the delivery of medication to the respiratory tract, in which the medication is delivered as a dry powder in a single-use, single-dose manner. In particular aspects, a simple dry powder inhaler has fewer than 10 working parts. In some aspects, the simple dry powder inhaler is a passive inhaler such that the dispersion energy is provided by the patient's inhalation force rather than through the application of an external energy source.

A “median particle diameter” refers to the geometric diameter as measured by laser diffraction or image analysis. In some aspects, at least either 50% or 80% of the particles by volume are in the median particle diameter range.

A “Mass Median Aerodynamic Diameter (MMAD)” refers to the aerodynamic diameter (different than the geometric diameter) and is measured by laser diffraction.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

Throughout this application, the term “about” is used to indicate that a value includes the inherent variation of error for the device, the method being employed to determine the value, or the variation that exists among the study subjects or experimental studies. Unless another definition is applicable, the term “about” refers to ±5% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “essentially free of” or “essentially free” is used to represent that the composition contains less than 1% of the specific component. The term “entirely free of” or “entirely free” contains less than 0.1% of the specific component.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Dosing of Tacrolimus

### A. Study Design

i. Part A

The study was a blinded, randomized, placebo-controlled, single dose, dose-escalation study. Thirty-two (32) healthy adult male and female (women of non-child bearing potential only) subjects were enrolled. Eight (8) subjects were assigned to each of four dose levels. Screening of subjects occurred within 28 days prior to the first dose administered.

Study subjects were randomized to receive either a placebo (2 subjects per group) or tacrolimus inhalation powder (6 subjects per group) by inhalation. The subject assignment were blinded to the investigator, the study coordinators, and the study subject. Subjects were automatically replaced if they discontinue the study prior to dosing. Dose levels were 0.5, 1.0, 2.5, and 5.0 mg. Dosing escalated in a sentinel fashion in the SAD portion only. On Day 1, two subjects assigned to the lowest dose cohort received a single dose of tacrolimus or placebo administered through the Plastiape® inhaler. Blood samples and safety measurements were collected over a 48-hour period (until Day 3) following the drug administration. If the drug is deemed safe by the TFF Medical Monitor and the Investigator after the review of safety information, the remaining subjects in the cohort were brought into the CRU no sooner than Day 4 for dosing on the next day. Blood samples and safety measurements were again collected over a 48-hour period.

Escalation did not proceed unless adequate safety is confirmed for the entire dosing cohort. The safety data were evaluated by the SMC who recommended whether dose escalation may proceed. The next highest dose cohort were not dosed for a minimum of 3 days following the completion of sampling and safety assessment from the last subject of the preceding cohort. Dosing continued in a sentinel fashion until either all cohorts are completed or significant adverse events (Grade 3 or greater) considered related to the drug or inhaler is observed in two or more subjects and/or stopping criteria are met. If any AEs were reported which meet stopping criteria and/or the SMC recommends against dose escalation, the next dosing cohort repeated the dose level used prior to that in which the adverse events were noted (i.e., the dose level lower to that in which the adverse events were present). Part A were terminated once that level has been completed.

Subjects randomized to receive the 2.5 mg inhalation dose participated in two administrations, with and without food. In this cohort, subjects were given the 2.5 mg inhalation dose on two separate occasions separated by at least 7 days. The administrations were not randomized; on the first dosing occasion, all subjects received the dose in a fasted state and on the second occasion the subjects received their dose after consuming a standardized high fat breakfast. The second occasion did not begin until the SMC has reviewed the safety data from the first occasion to ensure the safety of the subjects for the second dosing occasion. For the second occasion, the inhalation dose were delivered within 5 minutes after the breakfast is consumed. Blood samples were collected for PK determinations over a 48-hour period after each administration. Subjects were not replaced if they are discontinued for safety reasons pertaining to tacrolimus or the inhaler.

ii. Part B

The study is a blinded, randomized, placebo-controlled, multiple dose, dose-escalation study. Part B begins once the first three cohorts of Part A have completed and safety data has been reviewed. Twenty-four (24) healthy adult male and female (women of non-child-bearing potential only) subjects were enrolled. Eight (8) subjects were assigned to each of three dose levels (1.0 mg BID, 0.5 mg BID, or QD dose of either 1.0, 1.5, or 2.0mg, as determined by the SMC at the conclusion of Cohort 2 and after examining the safety and TDM data from that cohort) with six (6) subjects per cohort receiving tacrolimus inhalation and two (2) subjects receiving placebo.

Screening of subjects occurred within 28 days prior to the first dose administered. Study subjects were randomized to receive either a placebo or tacrolimus inhalation powder by inhalation. The subject assignment were blinded to the investigator, study coordinators and study subjects. Subjects were automatically replaced if they discontinue the study prior to dosing. On Day 1, subjects in each cohort begins receiving tacrolimus or placebo administered twice a day (every 12 hours) for Cohorts 1 and 2, and tacrolimus or placebo administered once per day (every 24 hours) for Cohort 3,through the Plastiape® inhaler. Blood samples and safety measurements were performed over 1 week as scheduled.

Escalation did not proceed to the next cohort unless safety is confirmed by the SMC for the entire dosing cohort. The next cohort did not begin dosing for a minimum of 3 days following the discharge of the last subject from the preceding cohort. Dosing continued until either all cohorts are completed or significant adverse events (Grade 3 or greater) considered related to the drug or inhaler is observed in two or more subjects or stopping criteria are met. The SMC could recommend that the next dosing cohort repeat the dose level used prior to that in which the significant adverse events were noted (i.e., the dose level lower to that in which the adverse events were present). Dose levels for Part B may be modified contingent upon the safety observed in the previous studies.

iii. Inclusion Criteria

Subjects fulfilled all of the following inclusion criteria to be eligible for participation in the study:
1. Healthy, adult, male or female (women of non-child bearing potential only), 18-65 years of age, inclusive, at screening. An attempt will be made to enroll female/male ratio=1:1.
2. Continuous non-smoker who has not used nicotine-containing products (including e-vaping) for at least 3 months prior to the first dosing and throughout the study, based on subject's self-reporting and urine cotinine levels at screening and check-in.
3. Body mass index (BMI) ≥18.0 and ≤32.0 kg/m2 at screening, and a minimum weight of at least 50.0 kg and a maximum weight of 120.0 kg at screening.
4. Medically healthy with no clinically significant abnormalities in medical history, physical and neurologic examination, laboratory profiles, vital signs or ECGs, as deemed by the PI or designee.
5. Female of non-childbearing potential must have undergone one of the following sterilization procedures (and have official documentation) at least 6 months prior to the first dosing:
   a. hysteroscopic sterilization;
   b. bilateral tubal ligation or bilateral salpingectomy;
   c. hysterectomy;
   d. bilateral oophorectomy;
   e. or be postmenopausal with amenorrhea for at least 1 year prior to the first dosing and follicle-stimulating hormone (FSH) serum levels greater than 40 mIU/mL consistent with postmenopausal status.
6. A non-vasectomized, male subject must agree to use a highly effective method of birth control with female partners of childbearing potential during the study and for 90 days following dosing. A highly effective method of birth control is defined as one that is associated with <1% failure rate when used correctly and consistently. For example:
   a. male subject must use of a condom; AND his female partner use of a highly-effective method of contraception:
      i. hormonal contraception associated with inhibition of ovulation, or
      ii. intrauterine contraceptive device,
   b. Sexual abstinence if this is consistent with participant's usual lifestyle
   No restrictions are required for a vasectomized male subject provided his vasectomy has been performed 4 months or more (and have official documentation) prior to Study Day 1. A subject who has been vasectomized less than 4 months prior to Study Day 1 or does not have official documentation of his vasectomy must follow the same restrictions as a non-vasectomized subject.
7. If male, must agree not to donate sperm from the first dosing until 90 days after the last dosing.
8. Agrees to abstain from recreational drug use throughout the study, from screening until follow-up.
9. Understands the study procedures in the informed consent form (ICF), and be willing and able to comply with the protocol.
10. Cockcroft-Gault Equation estimated creatinine clearance of ≥80 mL/min
11. Succeeds in training on the use of the device for up to 2 inhalations of empty capsule
12. Subjects must have an FEV1 ≥80%

iv. Exclusion Criteria

Subjects were not enrolled in the study if they meet any of the following criteria:
1. Is mentally or legally incapacitated or has significant emotional problems at the time of the screening visit or expected during the conduct of the study in the opinion of the PI or designee.
2. History or presence of clinically significant medical or psychiatric condition or disease in the opinion of the PI or designee.
3. History of any illness that, in the opinion of the PI or designee, might confound the results of the study or poses an additional risk to the subject by their participation in the study.
4. History of any illness that, in the opinion of the PI or designee, might confound the results of the study or poses an additional risk to the subject by their participation in the study.
5. Has a history of lung disease, asthma or reactive airway disease.
6. History or presence of alcoholism or drug abuse within the past 2 years prior to the first dosing.
7. History or presence of hypersensitivity or idiosyncratic reaction to tacrolimus, cyclosporine, or any chemically related compound (everolimus, sirolimus).
8. History of lactase deficiency
9. Has had surgery or any medical condition within 6 months prior to first dosing which may affect the absorption, distribution, metabolism, or elimination of the study drug, in the opinion of the PI or designee.
10. Female subjects of childbearing potential.
11. Female subjects with a positive pregnancy test or who are lactating.
12. Positive urine drug or alcohol results at screening or first check-in.
13. Positive cotinine results at screening.
14. Positive result at screening for tuberculosis (ie. positive result for QuantiFERON TB-Gold).
15. Positive results at screening for human immunodeficiency virus (HIV), hepatitis B surface antigen (HBsAg) or hepatitis C virus (HCV).
16. QTcF interval is >450 msec (males) or >470 msec (females) or has ECG findings deemed abnormal with clinical significance by the PI or designee at screening or prior to dosing on Day 1.
17. Seated blood pressure is less than 90/60 mmHg or greater than 140/90 mmHg at screening.
18. Seated heart rate is lower than 40 beats per minutes (bpm) or higher than 99 bpm at screening.
19. Unable to refrain from or anticipates the use of:
   a. Any drug, including prescription and non-prescription medications, herbal remedies, or vitamin supplements beginning 14 days prior to the first dosing and throughout the study. After first dosing, acetaminophen (up to 2 g per 24 hours) may be administered at the discretion of the PI or designee. Hormone replacement therapy will not be allowed.
   b. Any drugs known to be strong inhibitors and/or inducers of CYP3A4/5, for 28 days prior to the first dosing and throughout the study. Appropriate sources (e.g., Flockhart Table) will be consulted to confirm lack of PK/PD interaction with tacrolimus.
20. Donation or loss of 50 to 499 mL whole blood within 30 days or more than 499 mL whole blood within 56 days prior to the first dosing.
21. Plasma donation within 7 days prior to the first dosing.
22. Has coagulation test outside of normal ranges at screening or first check-in (confirmation of results may be done once).
23. Has platelet, hemoglobin, and hematocrit that are below the lower limit of normal at screening or first check-in (confirmation of results may be done once).
24. Has liver function tests including alanine aminotransferase (ALT), aspartate aminotransferase (AST), ALP and total bilirubin that are greater than the upper limit of normal at screening and first check in (confirmation of results may be done once).
25. Estimated Cockcroft-Gault creatinine clearance <80 mL/min at screening.
26. Participation in another clinical study within 30 days prior to the first dosing. The 30-day window will be derived from the date of the last blood collection or dosing, whichever is later, in the previous study to Day 1 of Period 1 of the current study.
27. Had a treatment with other investigational drug within 5 times the elimination half-life, if known (e.g., a marketed product) or within 30 days (if the elimination half-life is unknown), whichever is longer, prior to Study Day 1 dosing.
28. Has received any live virus vaccine within 6 weeks prior to this study or anticipates receiving a live virus vaccine within 6 months after study completion.
29. Demonstrates an inability to operate the inhalation device after training
30. Evidence of COVID-19 infection.

v. Study Assessments

For this study, the blood collections for tacrolimus concentration determinations are considered critical parameters. Consequently, these samples were collected as close to the scheduled time as possible. All other procedures should also be completed as close to the prescribed/scheduled times but can be staggered if necessary. Any procedure or collection should be documented with the precise clock time.

All entrance criteria related to study assessments (e.g., spirometry, clinical laboratory values, training on use of inhalation device, etc.) were reconfirmed on the day of check-in for each Cohort. Any nonscheduled procedures required for urgent evaluation of safety concerns took precedence over all routine scheduled procedures.

Within 28 days prior to the first dosing, medical history and demographic data, including sex, age, race, ethnicity, body weight (kg), height (cm), BMI (kg/m²) and history of tobacco use were reported. Each subject had a physical examination, complete neurologic examination, vital sign measurements (heart rate, blood pressure, temperature, and respiratory rate), 12-lead ECG, and the laboratory tests of hematological, coagulation, hepatic, and renal function and additional tests.

During screening, the subjects were provided a working model of the device and instructions on its use. The subjects were properly trained on how to use the inhalation device. If the subjects are unable to demonstrate proper use of the device, they were not allowed to enroll in the study.

Full physical examinations including complete neurologic examinations were performed. Symptom-driven physical examinations were performed at other times, if deemed necessary by the PI or designee.

Single measurements of body temperature, respiratory rate, blood pressure and heart rate, were measured. Additional vital signs could be taken at any other times, if deemed necessary. Vital signs were performed with subjects in a supine position, whenever possible.

Blood pressure, heart rate, respiratory rate, and body temperature were measured within 24 hours prior to Day 1 dosing in each period for the pre-dose time point. During Part B of the study all evening pre-dose vital signs and Day 2-6 morning pre-dose vital signs were taken within 45 minutes of the next dose. When scheduled post-dose, vital signs were performed within approximately 25 minutes of the Day 1, 12 hour timepoint, and 15 minutes of the remaining scheduled time points.

Single 12-lead ECGs were performed except for the pre-dose ECG performed prior to the initiation of dosing on Day 1. For the ECG prior to dosing on Day 1, two ECGs must be performed within 15 minutes and the QTcF values were averaged for the baseline pre-dose value. This baseline pre-dose average QTcF must be below the threshold in Exclusion Criterion 16 in order for the subject to be eligible for dosing. These Day 1 pre-dose ECGs were performed within 90 minutes of dosing. Additional ECGs may be taken at any other times, if deemed necessary by the PI or designee.

ECGs were performed at scheduled timepoints (±15 minutes) after subjects have remained in a supine position for approximately 5 minutes. All ECG tracings were reviewed by the PI or designee. All ECGs must contain the following elements: date and time of collection, position, heart rate, QT interval, PR interval, QRS interval, RR interval, QTcB and QTcF, and clinical interpretation.

Subjects had telemetric monitoring during the first 2 hours post dosing (Part A) and had telemetric monitoring during the first 2 hours after the 1st dose (Day 1) and after the 13ₜₕ dose (MAD Cohorts 1 and 2) or 7ₜₕ dose (MAD Cohort 3) on Day 7 in Part B. If an arrythmia is observed during telemetry, an unscheduled ECG with a prolonged rhythm strip were collected, read and interpreted by a board certified cardiologist, and recorded in the CRF. The Medical Monitor was notified should this occur. The QTc values are reviewed closely. Fridericia's correction for the QT interval (QTcF) are the primary endpoint for review.

Furthermore, body weight (kg) are reported.

Spirometry including forced expiratory volume in 1 second (FEV₁), percent predicted FEV₁, forced vital capacity (FVC), percent predicted FVC, forced expiratory flow at 25% to 75% of forced vital capacity (FEF_{25-75%}) and percent predicted FEF_{25-75%} were performed at the specified timepoints (±30 minutes). Pulse oximetry were monitored at specified timepoints (±30 minutes).

vi. Adverse Events

An AE means any untoward medical occurrence associated with the use of a drug in humans, whether or not considered drug related.

An Adverse Device Effect (ADE) means all untoward and unintended responses to the medical device. The phrase “responses to a medical device” means that a causal relationship between the device under investigation and an AE is at least a reasonable possibility, i.e., the relationship cannot be ruled out.

All cases judged by either the reporting medically qualified professional or the sponsor as having a reasonable suspected causal relationship to the device qualified as a device effect. This also includes any event resulting from insufficiencies or inadequacies in the instruction for use or deployment of the device and includes any event that is a result of a user error.

A Suspected Adverse Drug Reaction (ADR) means any AE for which there is a reasonable possibility that the drug caused the AE. Reasonable possibility means there is evidence to suggest a causal relationship between the drug and the AE. ADRs are a subset of all suspected adverse reactions for which there is reason to conclude that the drug caused the event. This would include all noxious and unintended responses to a medicinal product related to any dose. An AE can therefore be any unfavorable and unintended sign (including an abnormal laboratory finding), symptom, or disease temporally associated with the use of a medicinal (investigational) product, whether or not related to the medicinal (investigational) product. An unexpected ADR is one in which the nature or severity is not consistent with the applicable product information.

Serious Adverse Device Effects (SADE): A serious adverse device effect (SADE) is any untoward medical occurrence seen in a patient that can be attributed wholly or partly to the device which resulted in any of the characteristics or led to characteristics of a Serious adverse event. SADE is also any event that may have led to these consequences if suitable action had not been taken or intervention had not been made or if circumstances has been less opportune. All cases judged by either the reporting medically qualified professional or the sponsor.

Unanticipated Adverse Device Effect (UADE): Any serious adverse device effect on health or safety or any life-threatening problem or death caused by, or associated with a device, if that effect, problem, or death was not previously identified in nature, severity or degree of incidence in the investigational plan or application (including a supplementary plan or application), or any other unanticipated serious problem associated with a device that related to the rights, safety or welfare of the subject.

No device related events were assessed as “anticipated” as per Instructions for Use for the IFU RS01—Dry Powder Inhalation Device.

vii. Stopping Criteria

A Safety Monitoring Committee (SMC) monitored the safety and tolerability of tacrolimus. The SMC reviewed the safety and PK data as well as data integrity, and overall conduct of the trial. In the event that there are urgent medical concerns for any individual subject, the TFF Medical Monitor in conjunction with the Principal Investigator made any necessary decisions, including the decision to stop or not stop study drug treatment for a given subject, and notified the SMC about these actions accordingly. Repeats of any tests or assessments were done per the discretion of the Investigator and/or TFF Medical Monitor.

The SMC meet prior to dose escalation for both Part A and Part B studies as well as if deemed necessary by Medical Monitor of TFF Pharmaceutical, Inc., Principal Investigator and SMC Chairperson based upon SAE events. Any dose escalation were conducted based on unanimous agreement among SMC members, sponsor and Principal Investigator. The SMC was entitled to recommend at any time to modify the dose escalation to lower than scheduled doses or terminate a cohort or the entire study, especially if any of the following criteria are fulfilled:
1. An absolute FEV1 decline of ≥20% from baseline that is not considered low due to effort issues. The baseline values were compared as follows:
   a. For both Part A & B a comparison of pre dose FEV1 on Day 1 (0 Hr.) vs any post dose FEV1.
   b. For Part B, a comparison of pre dose on FEV1 Day 1 & Day 7 (0 Hr.) with post dose FEV1 on other individual time points on same day or any other unscheduled FEV1.
2. Subject has a marked prolongation of the QT/QTcF interval during treatment with the study drug. There are 2 rules for stopping treatment due to QTcF intervals:
   a. An absolute QTcF value of ≥500 msec at any time. The TFF Medical Monitor was notified immediately and subject discontinued from further treatment.
   b. An increase in QTcF on 2 consecutive ECGs performed 15 minutes apart that is above 450 msec for males or above 470 msec for females. The TFF Medical Monitor were notified immediately and the subject discontinued from further treatment.
      NOTE: Subjects with an increase from baseline in QTcF of >60 msec may or may not be removed from treatment. The Principal Investigator in consultation with the TFF Medical Monitor will make this decision and convey this decision to the SMC.

The baseline QTcF values will be compared as follows:
a. For both Part A & B, a comparison of the pre dose average QTcF on Day 1 (0 Hr.) vs all post dose ECGs.
b. For Part B, a comparison of the pre dose average QTcF on Day 1 and the QTcF on Day 7 (0 Hr., pre dose 13 for MAD Cohorts 1 and 2, pre dose 7 for MAD Cohort 3) with post dose ECG on other individual time points on the same day or any other unscheduled ECGs.
3. Subject has an AST and/or ALT increase ≥3×upper limit of normal (ULN) and/or total bilirubin increase ≥2×ULN during the study. NOTE: If this occurs, the subject will not receive any additional doses and should be followed with daily laboratory repeat testing until AST or ALT levels are <3×ULN and/or total bilirubin is <2×ULN, after which subjects will be followed up at a minimum weekly interval. This monitoring will be followed until levels have declined to normal levels or an abnormal value is judged not clinically significant by the Investigator.

For all subjects, venous blood samples will be collected in blood collection tubes containing K₂EDTA at scheduled time points was added for the determination of plasma tacrolimus concentration. PK blood draws will be taken within 15 minutes prior to dosing for the pre-dose collection. Post-dose blood draws will be performed within the following windows (Table 1) of the scheduled time points:

TABLE 1
Timing Windows

| Deviation Window Hour | Deviation Window |
|---|---|
| >0.0 to 8.0 hour | ±5 minutes |
| >8.0 to 24.0 hour | ±15 minutes |
| 48 hour or later samples | ±30 minutes |

Samples were processed according to the Laboratory Manual and shipped according to the site's standard operating procedures and instructions from the Sponsor or bioanalytical laboratory. Instruction for blood sampling, collection, processing, and sample shipment were provided separately.

PK parameters for plasma tacrolimus are calculated as follows, as appropriate:
AUC₀₋₂₄: The area under the concentration-time curve, from time 0 to the 24-hour time point, as calculated by the linear trapezoidal method. If the 24-hour plasma concentration is missing, below the limit of quantification or not reportable, then this parameter will not be calculated.
AUC₀₋ₜ: The area under the concentration-time curve, from time 0 to the last observed non-zero concentration, as calculated by the linear trapezoidal method.
AUC_{0-υ}: The area under the concentration-time curve, from time 0 to the last time of a dosing interval.
AUC_{-inf}: The area under the concentration-time curve from time 0 extrapolated to infinity. AUC_{0-inf} is calculated as the sum of AUC₀₋ₜ plus the ratio of the last measurable plasma concentration to the elimination rate constant.
AUC_{% extrap}: Percent of AUC_{0-inf} extrapolated, represented as (1−AUC₀₋ₜ/AUC_{0-inf})*100
Cₘₐₓ: Maximum observed concentration.
CL/F Apparent total body clearance after oral administration, calculated as Dose/AUC_{0-inf}.
Tₘₐₓ: Time to reach Cₘₐₓ. If the maximum value occurs at more than one time point, Tₘₐₓ is defined as the first time point with this value.
Kel: Apparent first-order terminal elimination rate constant calculated from a semi-log plot of the plasma concentration versus time curve. The parameter will be calculated by linear least-squares regression analysis using the maximum number of points in the terminal log-linear phase (e.g., three or more non-zero plasma concentrations).
t_{1/2}: Apparent first-order terminal elimination half-life are calculated as 0.693/Kel.
Vz/F: Apparent volume of distribution during the terminal elimination phase after oral administration, calculated as (Dose/AUC₀₋ᵢₙₜ)×Kel.

No value for Kel, AUC0-inf, AUC % extrap, t1/2, CL/F, or Vz/F will be reported for cases that do not exhibit a terminal log-linear phase in the concentration-time profile. No PK parameters will be calculated for subjects with 2 or fewer consecutive time points with detectable concentrations. Individual and mean plasma concentration time curves (both linear and log-linear) will be included in the final report. Dose proportionality will be tested across all doses used in Parts A and B, using standard power regression methods. T. will be analyzed using non-parametric analysis (Walsh averages and appropriate quartile of the Wilcoxon Signed Rank Test Statistic). Tmax will not be ln-transformed

viii. Tacrolimus Composition Used in the Studies

The tacrolimus drug product is Tacrolimus Inhalation Powder, for oral inhalation use. The tacrolimus drug product consists of capsules containing tacrolimus powder for oral inhalation and the use of the Plastiape Monodose dry powder inhaler device (RS01). For the human studies, tacrolimus capsules containing 0.5 mg or 2.5 mg of tacrolimus and lactose monohydrate and leucine in a concentration necessary to facilitate capsule filing and delivery and will be loaded in hypromellose capsules with a white opaque cap and a white opaque body. The placebo drug product consists of capsules containing lactose monohydrate only and the use of the same Plastiape Monodose dry powder inhaler device (RS01). The composition of Tacrolimus Inhalation Powder 0.5 mg and 2.5 mg are provided in Table 2 and Table 3, respectively.

TABLE 2
Composition of Tacrolimus Inhalation Powder, 0.5 mg

| | | | | Reference |
|---|---|---|---|---|
| Ingredient | Composition | | Function | to Standard |
| Quantity (mg) | | | Percent | |
| Tacrolimus | 0.5 | 10 | Drug Substance | USP |
| Purified Water | | | | |
| Lactose | 2.0 | 40 | Diluent | NF |
| monohydrate | 2.5 | 50 | Diluent | USP |
| Leucine | Removed during processing | | Processing Aid | In House |
| Acetonitrile | Removed during processing | | Processing Aid | USP |
| Total | 5 | | 100 | |
| Empty Hard | 1 ea | — | Dosage Form | USP/NF |
| Hypromellose | | | Encapsulation | |
| Capsule, Size | | | | |
| 3, White₁ | | | | |

₁The white, empty hard hypromellose capsule shell is composed of titanium dioxide and hypromellose. Although the capsule shell is not a compendial item, it is composed of compendial materials that are tested to the current compendium.

TABLE 3
Composition of Tacrolimus Inhalation Powder, 2.5 mg

| | | | | Reference to |
|---|---|---|---|---|
| Ingredient | Composition | | Function | Standard |
| Quantity (mg) | | | Percent | |
| Tacrolimus | 2.5 | 50 | Drug Substance | USP |
| Lactose | 2.5 | 50 | Diluent | NF |
| monohydrate | | | | |
| Acetonitrile | Removed during processing | | Processing Aid | In House |
| Purified Water | Removed during processing | | Processing Aid | USP |
| Total | 5 | 100 | | |
| Empty Hard | 1 ea | — | Dosage Form | USP/NF |
| Hypromellose | | | Encapsulation | |
| Capsule, Size 3, | | | | |
| White₁ | | | | |

₁The white, empty hard hypromellose capsule shell is composed of titanium dioxide and hypromellose. Although the capsule shell is not a compendial item, it is composed of compendial materials that are tested to the current compendium.

The composition of Placebo Inhalation Powder is provided in Table 4.

TABLE 4
Composition of Placebo Inhalation Powder

| | | | | Reference to |
|---|---|---|---|---|
| Ingredient | Composition | | Function | Standard |
| Quantity (mg) | | | Percent | |
| Lactose Monohydrate | 5 | 100 | Diluent | NF |
| Total | 5 | 100 | | |
| Empty Hard | 1 each | | Dosage Form | USP/NF |
| Hypromellose Capsule, | | | Encapsulation | |
| Size 3, White₁ | | | | |

₁The white, empty hard hypromellose capsule shell is composed of titanium dioxide and hypromellose. Although the capsule shell is not a compendial item, it is composed of compendial materials that are tested to the current compendium.

The manufacturing process for the 2.5 mg capsules of Tacrolimus Inhalation Powder started with the preparation of a 50:50 (w/w) tacrolimus/lactose monohydrate powder using thin film freezing. The thin film freezing process starts by dissolving the required amount of tacrolimus and lactose monohydrate in a cosolvent mixture of acetonitrile and water. The solution is then frozen by dropwise addition to the surface of a rotating drum cooled with liquid nitrogen. The frozen solution is collected and dried by lyophilization to remove acetonitrile and water. The level of acetonitrile is controlled to not more than 410 parts per million (ppm) per ICH Q3C. The 50% tacrolimus powder for inhalation is amorphous as characterized by x-ray diffraction.

The Tacrolimus Inhalation Powder 0.5 mg drug product was packaged for clinical use in white high density polyethylene (HDPE) bottle with polypropylene (PP) closures containing a silica gel desiccant for storage at 36° F. to 46° F. (2° C. to 8° C.).

The Tacrolimus Inhalation Powder 2.5 mg drug product and the placebo drug product were packaged for clinical use in white high density polyethylene (HDPE) bottle with polypropylene (PP) closures containing a silica gel desiccant for storage in a dry place at 68° F. to 77° F. (20° C. to 25° C.); excursions permitted to 59° F. to 86° F. (15° C. to 30° C.).

Tacrolimus Inhalation Powder and Placebo Inhalation Powder was used with the Plastiape Monodose inhaler. The drug product inhalation (DPI) device is manufactured by Plastiape S.p.A., located in Osnago, Italy. Plastiape has filed U.S. DMF No. 17864 for the RS01 device. The high resistance version of the RS01 device was used with Tacrolimus Inhalation Powder. The Plastiape Monodose DPI device was used in Bronchitol® (mannitol powder for inhalation) and Aridol® (mannitol) that are both currently approved in Australia. The Plastiape inhaler consists of a white protective cap and a base with mouthpiece, capsule chamber, and 2 push buttons.

The in vitro aerosol performance of Tacrolimus Inhalation Powder 0.5 mg and 2.5 mg was characterized using a next generation impactor (NGI) according to USP <601> per c.6.1—Apparatus 5. For the RS01 high resistance device to be used with the inhalation powder capsules, the flow rate was ˜60 L/min to achieve a pressure drop of 4 kPa. Table 5 compares the aerodynamic properties of Tacrolimus Inhalation Powder 0.5 mg and 2.5 mg from representative development batches of drug product.

TABLE 5
Aerodynamic Properties of Tacrolimus Inhalation Powder
at 60 L/min Using a Plastiape Monodose RS01 High
Resistance Dry Powder Inhaler Device Product

| Dose | 0.5 mg | 2.5 mg |
|---|---|---|
| MMAD (μm) | 2.08 (0.10)₁ | 1.75 (0.09) |
| GSD | 4.84 (0.26) | 2.37 (0.27) |
| FPF (% of Recovered Dose) | 55.47 (2.96) | 81.07 (1.82) |
| FPF (% of Delivered Dose) | 61.61 (3.07) | 84.64 (2.23) |
| Emitted Dose (%) | 90.03 (0.66) | 95.79 (0.80) |

MMAD = Mass Median Aerodynamic Diameter
GSD = Geometric Standard Deviation
FPF = Fine Particle Fraction
₁Mean Value (Standard Deviation)

### B. Pharmacokinetic Analysis

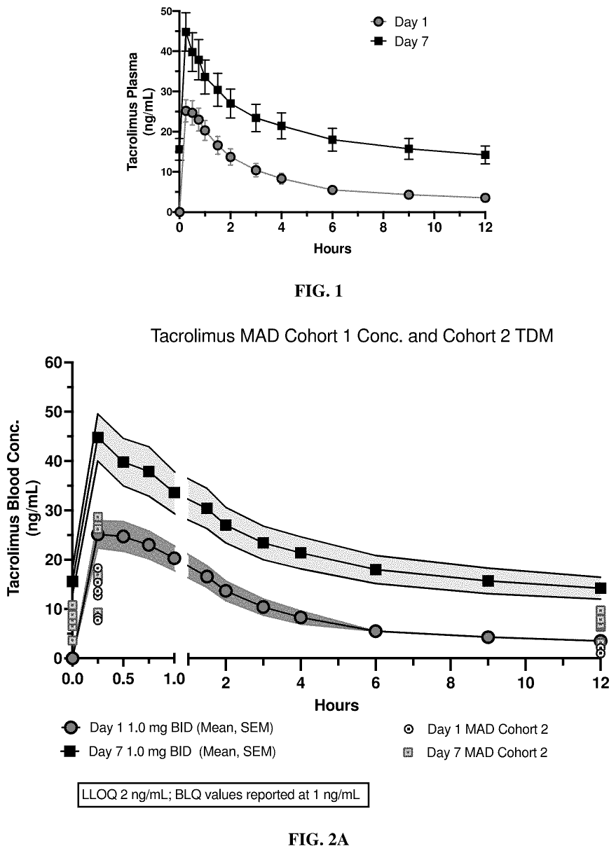

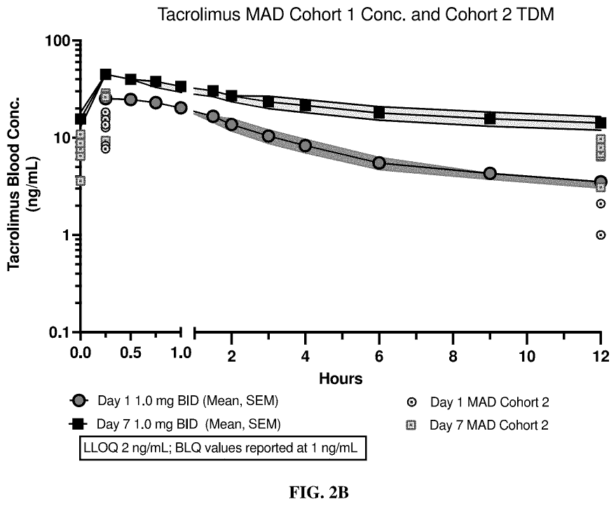

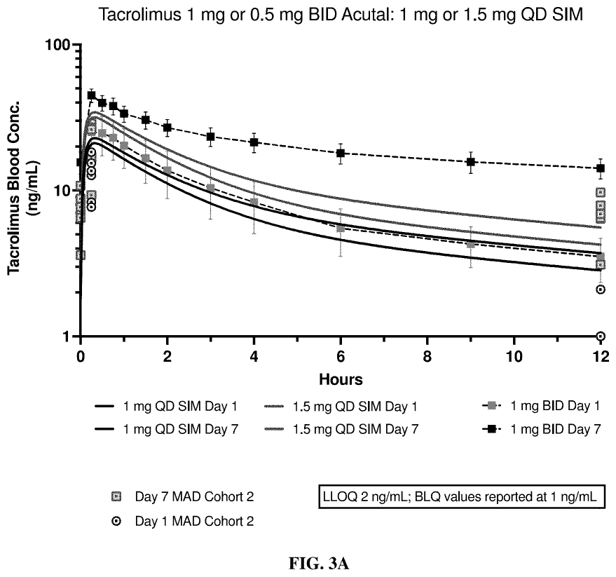

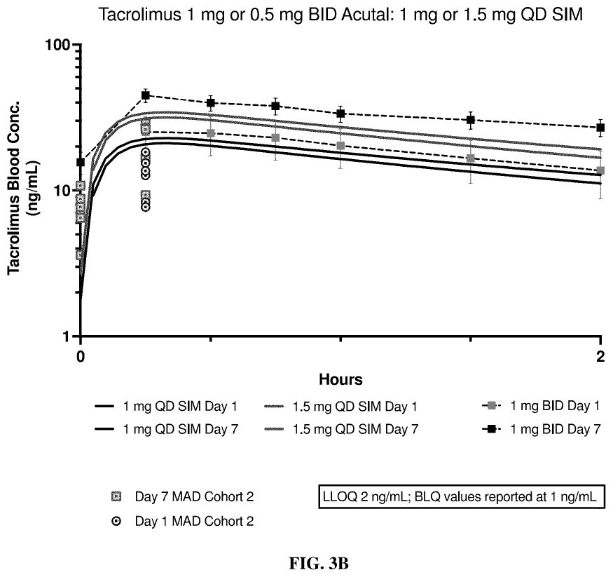

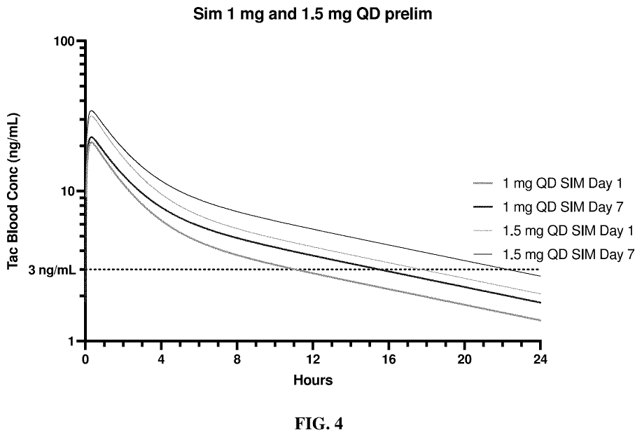

Recent data from healthy volunteers (FIG. 1) from the 1 mg BID repeat dose group generally were predicted from the oral PK model with 75% F (absorption) that was developed to simulate systemic exposure of tacrolimus after pulmonary inhalation. See FIGS. 2A & 2B. The actual arithmetic mean of 45 ng/mL was in agreement with the simulated geometric mean exposure of slightly greater than 45 ng/mL. Actual trough values at 12 hours post dose on Day 1 and Day 7 were closer to the upper 90% confidence interval for simulated exposure. This preliminary pharmacokinetic data suggests that it is feasible to deliver low mg doses of tacrolimus by inhalation and achieve similar concentration values at trough within the TDM concentration range of 10-15 ng/mL at a lower total daily dose. See FIGS. 3-4. More importantly, the Cₘᵢₙ (trough) concentrations at 12 h are slightly higher than predicted from the oral PK model and, just as important, observed Cₘₐₓ is lower than predicted. This inverse relationship of lower peak concentration and higher trough concentration provides for a more consistent exposure (reduced swing) and 1) may improve patient compliance with reduced GI AEs because of the route of administration, 2) provide higher TDM Cₘᵢₙ trough concentrations at lower mg doses, and 3) may indicate that once-a-day dosing as a feasible dosing strategy with a lower drug burden.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
U.S. Pat. No. 8,968,786
U.S. Patent App. No. 2010/0221343
Beinborn et al., European journal of pharmaceutics and biopharmaceutics: Official Journal of Arbeitsgemeinschaft fur Pharmazeutische Verfahrenstechnik e V.; 81(3):600-8, 2012a.
Beinborn et al., Int J Pharmaceut.; 429(1-2):46-57, 2012b.
Brunet et al., Therapeutic Drug Monitoring, 41(3):261-307, 2019.
Calne et al., Lancet, 2:1323-1327, 1978.
Carvalho et al., European journal of pharmaceutics and biopharmaceutics: Official Journal of Arbeitsgemeinschaft fur Pharmazeutische Verfahrenstechnik e V.; 88(1):136-47, 2014.
Chen and Prasad, Pharmgenomcis Pers. Med., 11:23-33, 2018.
Christie et al., J. Heart Lung Transplant., 29:1104-1118, 2010.
Christie et al., J. Heart Lung Transplant., 31:1073-1086, 2012.
DiNunzio et al., Mol Pharm., 5(6):968-80, 2008.
Egan et al., Am. J. Transplant, 6:1212-1227, 2006.
Engstrom et al., Pharm Res.; 25(6):1334-46, 2008.
Gilljam et al., J. Cyst. Fibros., 9:272-276, 2010.
Handbook of Pharmaceutical Salts: Properties, and Use (P. H. Stahl & C. G. Wermuth eds., Verlag Helvetica Chimica Acta, 2002).
Helmi et al., Chest, 123:800-808, 2003.
Iwasaki et al., Drug Metab. Pharmaockinet., 22(5):328-35, 2007.
Lang et al., Mol Pharm.; 11(1):186-96, 2014a.
Lang et al., J Drug Deliv Sci Tec.; 24(2):205-11, 2014b.
Liu et al., European journal of pharmaceutics and biopharmaceutics: official journal of Arbeitsgemeinschaft fur Pharmazeutische Verfahrenstechnik e V.; 96:132-42, 2015.
Machuca et al., Curr. Opin. Organ Transplant, 17:479-484, 2012.
O'Donnell et al., Drug Dev Ind Pharm.; 39(2):205-17, 2013.
Overhoff et al., Pharm Res.; 25(1):167-75, 2008.
Overhoff et al., European journal of pharmaceutics and biopharmaceutics: official journal of Arbeitsgemeinschaft fur Pharmazeutische Verfahrenstechnik e V.; 65(1):57-67, 2007b.
Overhoff et al., Int J Pharmaceut.; 336(1):122-32, 2007a.
Overhoff et al., J Drug Del Sci Tech.; 19(2):89-98, 2009.
Patterson et al., Ann. Thorac. Surg., 45:626-633, 1988.
Purvis et al., Aaps Pharmscitech.; 8(3):E58, 2007.
Sinswat et al., European journal of pharmaceutics and biopharmaceutics: official journal of Arbeitsgemeinschaft fur Pharmazeutische Verfahrenstechnik e V.; 69(3):1057-66, 2008.
Studer et al., Eur. Respir. J., 24:674-685, 2004.
Thakkar et al., Hum Vaccin Immunother.; 13(4):936-46, 2017.
Wang et al., Aaps Pharmscitech.; 15(4):981-93, 2014.
Watts et al., Int J Pharmaceut.; 384(1-2):46-52, 2010.
Watts et al., Pharm Res.; 30(3):813--25, 2013.
Yang et al., Int J Pharmaceut.; 361(1-2):177-88, 2008.
Zhang et al., European journal of pharmaceutics and biopharmaceutics: official journal of Arbeitsgemeinschaft fur Pharmazeutische Verfahrenstechnik e V.; 82(3):534-44, 2012.

What is claimed is:

1. A method of modulating an immune response in a patient in need thereof, comprising administering by inhalation to the lungs of the patient an appropriate amount of a dry powder composition of drug particles comprising tacrolimus, the patient being administered a dose once during a 24 hour period sufficient to generate a blood concentration of tacrolimus in the patient of at least 3 ng/mL at a time point of 24 hours after the administration once the patient has been administered the dose for 3 consecutive days.

2. The method of claim 1, wherein the patient has been administered the dose for 7 consecutive days.

3. The method of claim 1, wherein the patient has not been administered any additional tacrolimus other than the dose.

4. The method of claim 1, wherein the dose is administered using a single inhaler capsule.

5. The method of claim 1, wherein the dose is administered using multiple inhaler capsules of an appropriate amount on a single occasion.

6. The method of claim 1, wherein the blood concentration of tacrolimus is at least 5 ng/mL at a time point of 24 hours after the administration.

7. The method of claim 1, wherein the blood concentration of tacrolimus is from about 3 ng/mL to about 15 ng/mL at a time point of 24 hours after the administration.

8. The method of claim 7, wherein the blood concentration of tacrolimus is from about 3 ng/mL to about 12 ng/mL at a time point of 24 hours after the administration.

9. The method of claim 8, wherein the blood concentration of tacrolimus is from about 3 ng/mL to about 7.5 ng/mL at a time point of 24 hours after the administration.

10. The method of claim 1, wherein the composition comprises a sugar.

11. The method of claim 10, wherein the sugar is lactose.

12. The method of claim 10, wherein the drug particles comprise tacrolimus and the sugar in a weight ratio of 5:1 to about 1:20.

13. The method of claim 12, wherein the weight ratio is from about 1:1 to about 1:10.

14. The method of claim 13, wherein the weight ratio is from about 1:2.5 to about 1:10.

15. The method of claim 1, wherein the composition comprises a dose of tacrolimus from about 0.05 mg to about 3.5 mg.

16. The method of claim 15, wherein the dose of tacrolimus is from about 0.1 mg to about 3.0 mg.

17. The method of claim 16, wherein the dose of tacrolimus is from about 0.25 mg to about 2.5 mg.

18. The method of claim 17, wherein the dose of tacrolimus is about 1.5 mg.

19. The method of claim 18, wherein the tacrolimus is in the amorphous form.

20. The method of claim 19, wherein at least 90% of the tacrolimus is in the amorphous form.

21. The method of claim 20, wherein at least 95% of the tacrolimus is in the amorphous form.

22. The method of claim 21, wherein at least 98% of the tacrolimus is in the amorphous form.

23. The method of claim 22, wherein at least 99% of the tacrolimus is in the amorphous form.

24. The method of claim 1, wherein the drug particles have a mass median aerodynamic diameter (MMAD) from about 0.5 μm to about 5.0 μm.

25. The method of claim 24, wherein the MMAD is from about 1.0 μm to about 3.5 μm.

26. The method of claim 25, wherein the MMAD is from about 1.5 μm to about 2.5 μm.

27. The method of claim 1, wherein the drug particles have a geometric standard deviation (GSD) from about 0.5 to about 8.

28. The method of claim 27, wherein the GSD is from about 1 to about 6.

29. The method of claim 28, wherein the GSD is from about 2 to about 5.
